(19) United States
(12) Patent Application Publication
(10) Pub. No.: US 2003/0004065 A1
(43) Pub. Date: Jan. 2, 2003

(54) Method for control of plant pathogens using a silver ion aqueous medium

(76) Inventor: Derek Belmonte, Paso Robles, CA (US)

Correspondence Address: FITCH EVEN TABIN AND FLANNERY, 120 SOUTH LA SALLE STREET, SUITE 1600, CHICAGO, IL, 60603-3406, US

(21) Appl. No.: 09/992,539

(22) Filed: Nov. 19, 2001

Related U.S. Application Data
(60) Provisional application No. 60/298,553, filed on Jun. 16, 2001.

(51) Int. Cl. A01N 55/02; C12N 5/00; C12N 5/02; A01N 59/16

(52) U.S. Cl. 504/152; 424/618; 435/420; 435/431

(57) ABSTRACT

The present invention is directed to a method of using electrically generated silver ions to control plant pathogens. Silver ions electrically generated in an aqueous medium and contacted with plant pathogens are effective for treating and preventing infections in plants caused by plant pathogens.

The present application is a non-provisional application claiming priority under 35 USC 119(e) to U.S. Provisional Application No. 60/298,553, of Belmonte et al., entitled METHOD FOR CONTROL OF PLANT PATHOGENS USING SILVER ION LIQUID SOLUTION, filed Jun. 16, 2001, which is incorporated herein in its entirety by reference.

### FIELD OF INVENTION

This present invention is directed to a method for the treatment and prevention of infections in plants caused by plant pathogens. More specifically, the invention is directed to the use of an electrically generated silver ion aqueous medium which is effective for treating and preventing infections in plants caused by plant pathogens.

### BACKGROUND

Protection of agriculturally important crops from disease has become a major concern in the agricultural industry. Plant pathogens cause disease by weakening the plant by absorbing food from the plant cells, secreting toxins, enzymes, or growth regulating substances that disturb or kill the plant cells, or by blocking the transport of food nutrients or water in the plant. The roots, stems, leaves, flowers, or fruits can be infected. The affected cells and tissues are weakened or destroyed, and cannot perform normal physiological functions, resulting in reduction of plant growth or death, and reducing crop quality or yield. The major causes of plant diseases includes bacteria, viruses, and fungi.

Plant pathogenic bacteria cause a variety of plant disease symptoms including fruit rot, galls, wilts, blight, and leaf spots. As bacteria multiply quickly, controlling them early in the disease process is critical. Copper and streptomycin compounds are the only chemical compounds currently available for the control of bacterial diseases in plants.

Fungal damage to plants can be caused by a fungus of genera such as Alternaria; Ascochyta; Botrytis; Cercospora; Colletotrichum; Diplodia; Erysiphe; Fusarium; Gaeumanomyces; Helminthosporium; Macrophomina; Nectria; Peronospora; Phoma; Phymatotrichum; Phytophthora; Plasmopara; Podosphaera; Puccinia; Pythium; Pyrenophora; Pyricularia; Rhizoctonia; Scerotium; Sclerotinia; Septoria; Thielaviopsis; Uncinula; Venturia; and Verticillium. Therefore, fungicidal compounds are not always effective because activity may be limited to a few species.

Fungus infection is a particular problem in damp climates and may become a major concern during crop storage. Plants have developed a certain degree of natural resistance to pathogenic fungi. Modern growing methods, harvesting and storage systems frequently provide a favorable environment for plant pathogens.

Virus diseases of cultivated plants cause substantial reductions in food, forage and fiber throughout the world. Control of these diseases has been based primarily on cultural practices that include removal of viral infected debris, eradication of weed hosts (herbicide applications), prevention of vector transmission (pesticide applications), indexing for virus-free starting material (seed or vegetative propagules) and breeding for disease resistance. Large scale methods for curing plants once they have become virus infected do not exist. Thus, the control of viral diseases is dependent upon methods to prevent or delay the establishment of infection.

It is well known that silver has germicidal properties. In fact, silver was employed as a germicide and antibiotic before modern antibiotics were developed. Nearly a century of experience has demonstrated the effectiveness of silver metal and silver salts against infection. Bolton in 1894 and Halstead in 1913 described the use of silver foil on fresh wounds to inhibit the growth of microorganisms, and argerol and silver nitrate were common bactericidal agents a decade or two ago.

Spadero in 1974 showed this highly oxidizing ion to be the effective germicidal agent, demonstrated a much higher concentration of the ion by anodically corroding metallic silver, and reported killing a broad spectrum of animal bacterial with as little as 400 nanoamperes of anodic DC current.

In typical agricultural practices, chemicals used for disease treatment in plants may be synthesized in one location, formulated in another, transported to a site of use, and then applied. Specific compounds which have activity against a certain bacteria, fungus or virus, are typically synthesized at a manufacturing facility. These compounds often have activity against only one or one group of pathogens. The active compounds are often provided to formulators who then formulate a product with the active compound and then sell that formulation to a distributor. The distributor provides the formulation to the farmer. The farmer may need to apply a particular formulation using applications methods that he might not normally due to the toxic properties of the chemicals being applied. Further, treatment of different types of pathogens may require separate applications of different formulations.

### SUMMARY

The present invention is directed to a method for treating and preventing infections in plants caused by a variety of plant pathogens. The method of the invention provides an electrically generated silver ion solution that is effective for treating plants that are already infected with a plant pathogen. The electrically generated silver ion solution is also effective for preventing infections or reinfections of the plant by a plant pathogen.

The present invention provides a number of advantages over traditional methods and chemicals used to treat plant infections. In an important aspect, the apparatus of the invention can be used to generate silver ions at the location of the plants, such as for example in a source of irrigation water being applied to the plants, thus eliminating the need to manufacture, formulate and ship treatment solutions to a field site and further eliminating the need for special application methods. In another important aspect, the electrically generated silver ion solution does not harm the host plant. The silver ion solutions of the invention are only germicidally active against bacteria, fungi and viruses. Host plants are not damaged by the silver ion solution and the general health and growth of the host plant is enhanced. The electrically generated silver ion solution is environmentally acceptable as the silver ion solution is not toxic to animals or humans. Any residues of electrically generated silver ions remaining on the plant are not toxic to animals or humans who consume the plant.

The present invention provides a method and apparatus for killing plant pathogens, such as for example bacteria, fungus, and viruses by providing an electrically generated silver ion aqueous solution. The silver ions are generated by slow electrical anodic corrosion of a silver wire located in the aqueous medium. In particular, a silver anode and a cathode of noncorroding metal are located in an aqueous medium, in particular (water), and a direct voltage is applied to the anode and cathode by passing a positive current in a voltage range into the silver anode in an amount effective for causing it to corrode slightly and give off silver ions which produces a germicidal aqueous solution. In this aspect, the device of the invention is effective for producing at least about 15 ppm silver ions in the liquid medium. The silver ion aqueous medium may be applied to crops, plants, cactus, trees, or vegetation through known and existing distribution systems such as, irrigation, sprinklers, tank sprayers, aerial applications, or manual application directly on the plant or by any other liquid distribution system.

In one aspect of the invention, silver ions are generated in a liquid medium and the liquid medium may be applied to the plant. In accordance with this aspect of the invention, the apparatus of the invention is placed into the liquid medium, such as for example an irrigation pond or water tank. Silver ion are electrically generated and the water may then be applied to the plant, such as for example through an irrigation ditch or by spraying.

In another aspect of the invention, silver ions are continuously generated in a flow through system. In accordance with this aspect of the invention, a liquid medium is continuously flowed through an apparatus that generates silver ion in the liquid medium. The liquid medium containing silver ions may then be applied to plants.

In an important aspect, the present invention provides a method for treating and preventing Pierce's disease and/or leaf roll virus. In accordance with this aspect of the invention, silver ions are electrically generated in a liquid medium. The liquid medium may be applied to plants having Pierce's Disease in an amount effective to reduce the occurrence of Pierce's Disease and/or leaf roll virus in the plant. The liquid medium may also be applied to plants to prevent the occurrence of Pierce's Disease and/or leaf roll virus.

### DESCRIPTION OF DRAWINGS

FIG. 1 shows one aspect of the apparatus of the present invention which is effective for generating a liquid medium containing silver ions.

FIG. 2 shows one aspect of the apparatus of the present invention which is effective for continuously generating a liquid medium containing silver ions.

FIG. 3 illustrates one aspect of the present invention with solar panels.

FIG. 4 illustrates general strategies for generating and applying a liquid medium containing silver ions to plants.

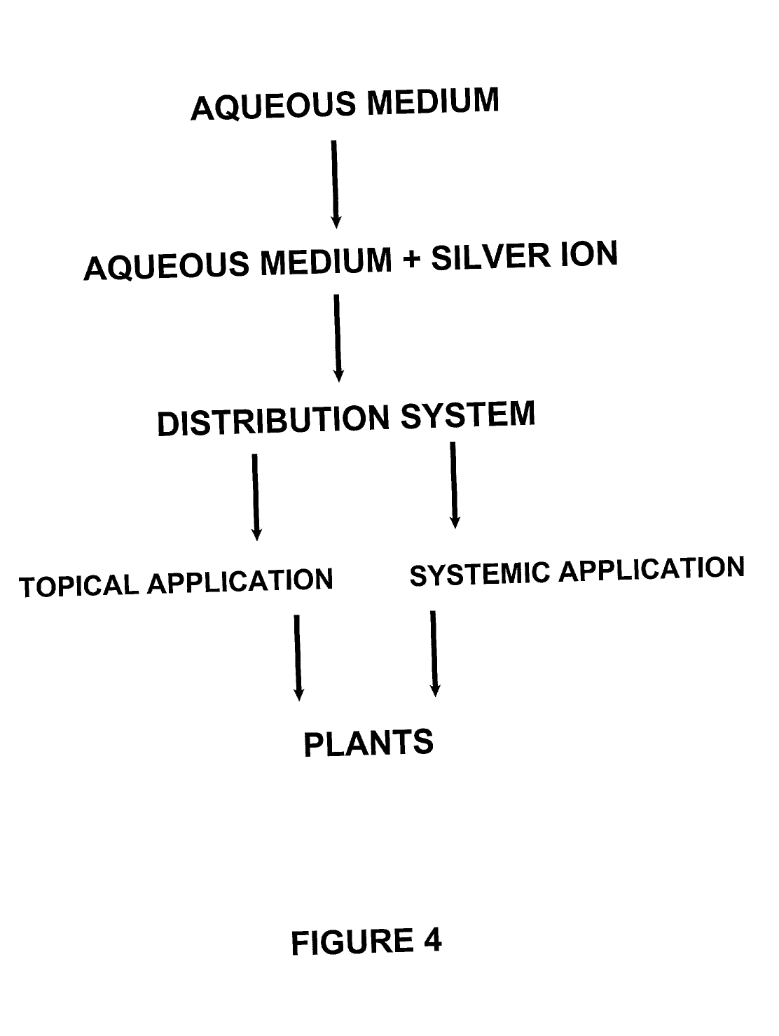

### DETAILED DESCRIPTION

The method of the present invention is effective for providing a silver ion solution in an aqueous medium at a concentration of at least about 15 ppm silver up to a concentration of about 300 ppm silver. The germicidal action of silver is not disease specific but is specific to bacteria, fungi and viruses. The application of electrically generated silver ion solution of the present invention to plants is effective for killing or inactivating plant pathogens either on contact or shortly after contact. Continued application of the electrically generated silver ion medium of the invention is effective for preventing infections or reinfections of plant pathogens and for allowing plants to be naturally restored to a fully functional state. Extended applications of the electrically generated silver ion medium of the invention do not result in development of resistance as with antibiotics. Further, applications of the electrically generated silver ion solution of the invention are safe, as silver is not considered to be toxic and does not effect or change the flavor of the end product.

As used herein, “treating infections in plants caused by plant pathogens” means applying an amount and concentration of aqueous medium containing silver ion that is effective for reducing the number of pathogenic organisms infecting a plant by at least about one log, and in an important aspect by about 3 logs or more.

As used herein, “preventing infections in plants caused by plant pathogens” means applying an amount and concentration of aqueous medium containing silver ion that is effective for preventing an increase in numbers of plant pathogens infecting a plant by more than about one log.

Plant Pathogens

The present invention provides a method for treating and preventing infections by plant pathogens. As used herein “plant pathogens” refers to bacteria, fungus and viruses that are known to infect plants.

Examples of bacteria that may be treated with the electrically generated silver ion solution of the present invention include Pseudomonas aeruginosa, Pseudomonas syringae, Pseudomonas viridiflava, Xanthomonas campestris pv. asclepiadas, Xyella fastidiosa, Acidovorax albilineans, and Acidovorax avenae sspl citrulli, E. coli, Erwinia amylovora and Ralstonia solanacearum. Diseases caused by these bacteria include fruit rot, galls, wilts, blight, and leaf spots.

In a very important aspect, the present invention is effective for treating and preventing infections caused by Xyella fastidiosa. This microorganism is know as the causative agent of Pierce's Disease. In the aspect of the invention, a liquid medium containing at least 15 ppm silver ion is applied to a plant as part of a normal watering schedule. For example in grapes, a volume of about 7 to about 10 gallons per acre may be applied per day, with half being applied during the day and half at night.

The electrically generated silver ion solution of the present invention is also effective for treating fungal infections. Fungal species which can be effectively treated by the electrically generated silver ion solution of the present invention include fungus of genera such as Alternaria, Ascochyta, Botrytis, Cercospora, Colletotrichum, Diplodia, Erysiphe, Fusarium, Gaeumanomyces, Helminthosporium, Macrophomina, Nectria, Peronospora, Phoma, Phymatotrichum, Phytophthora, Plasmopara, Podosphaera, Puccinia, Pythium, Pyrenophora, Pyricularia, Rhizoctonia, Scerotium, Sclerotinia, Septoria, Thielaviopsis, Uncinula, Venturia, Verticillium, and leaf roll virus. Plant disease caused by fungi include pre- and post-emergence seedling damping-off, hypocotyl rots, root rots, crown rots, vascular wilt, and other symptoms.

As used herein, “pathogenic plant virus” means a virus which infects plants and produces a condition considered to be abnormal and detrimental to the plant. Such viruses would typically consist of a genomic nucleic acid enclosed by coat protein subunits assembled around the nucleic acid in a specific geometric conformation, which are partially or completely removed during the disassembly phase of infection to expose the nucleic acid. Examples of plant viruses that can be treated with the silver ion solution of the present invention include tobacco mosaic virus (TMV), cucumber mosaic virus (CMV), cucumber green mottle mosaic virus (as CGMMV), potato virus X (PVX), lettuce mosaic virus (LMV), melon necrotic spot virus (MNSV) and the like.

Silver Ion

“Silver ion” as used herein refers to an atom or group of atoms that is not electrically neutral but carry a charge. The silver ions generated by the present invention are not initially colloidal or are non-colloidal. Colloidal refers to substances that diffuse through water at an exceedingly low rate in comparison with crystalline substances such as sodium chloride, sugar, and glycerol. Colloids typically have an amorphous shape or aggregation and hence, do not have a definite form, or a distinct crystalline structure.

True colloids, such as silver colloids, do not diffuse through water as quickly as an ion in water. As a result, a silver colloid is not as bioavailable or able to enter a cell as easily as a silver ion in solution. Hence, colloidal silver is not as effective as a silver ion in solution.

Electrical Generation of Silver Ions

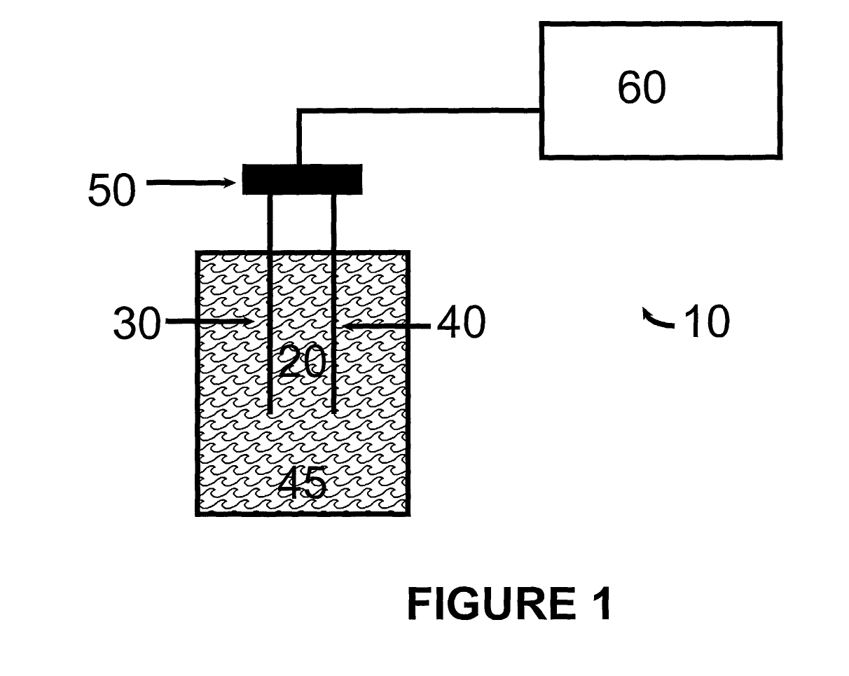

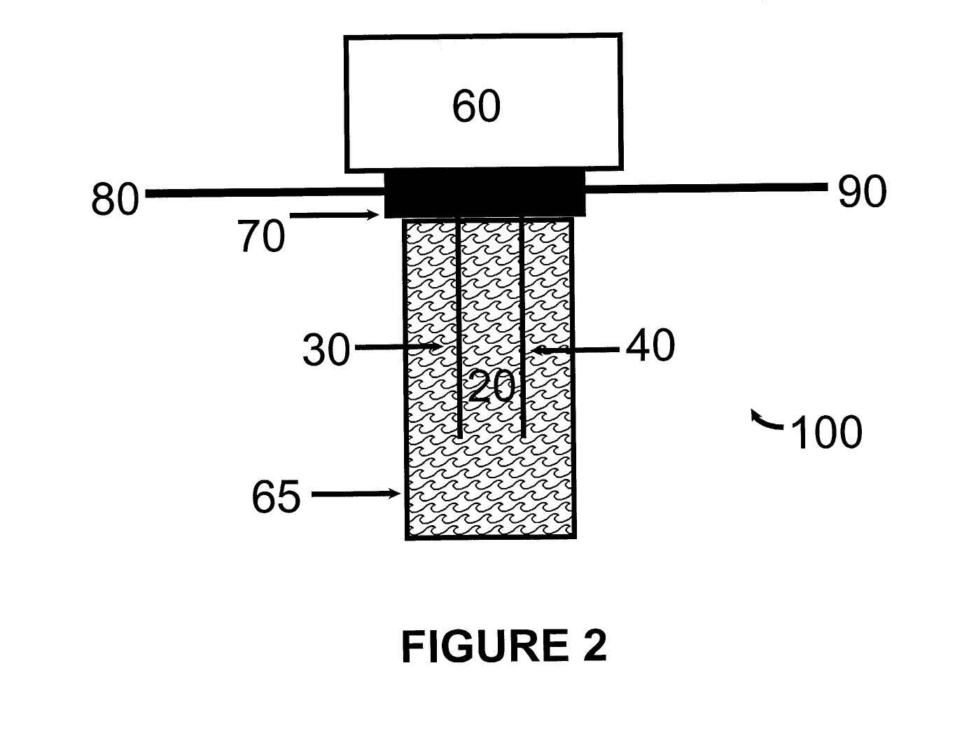

Examples of an apparatus effective for generating the silver ion solution of the present invention is generally illustrated in FIGS. 1 and 2.

As shown in FIG. 1, the apparatus 10 includes at least one set of electrodes 20. Each electrode set 20 includes a silver anode 30 and a cathode of noncorrosive metal 40. The anode and cathode may each be a wire that extends into a liquid medium 45. Each electrode set 20 extends down from a manifold 50.

Each set of electrodes 20 is electrically connected to a power transformer 60. The silver ion solution of the invention is provided by applying a direct voltage to the anode 30 and cathode 40 by passing a positive current in a voltage range of about 6 to about 30 volts to the silver anode 30, causing it to corrode slightly and give off silver ions. In this aspect of the invention, the quantity of electricity passed through the electrodes and the amount of silver ion produced may be determined from Faraday's Law. Hence, one of ordinary skill in the art can determine how quickly a desired concentration of silver ion concentration can be attained depending on the voltage used and the volume of liquid medium being treated. The power transformer 60 may be connected to electrical power, may be a battery, or may be a solar powered device.

The apparatus 10 as shown in FIG. 1 may be placed into a water container, an irrigation pond, a well or any other structure in which might contain an aqueous medium. The manifold 50 may be made of materials that will float on a liquid medium or the entire apparatus may be physically suspended in the liquid medium. This allows the silver ions to be generated in the location of their use and eliminates the need to transport the silver ion medium to a location where it is being used.

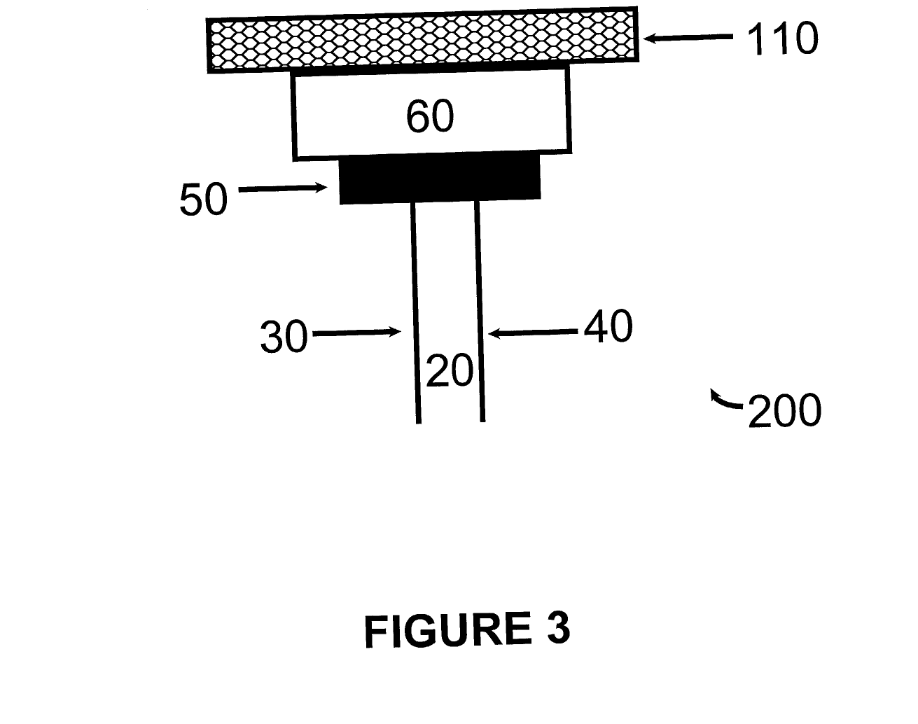

In another aspect of the invention as shown in FIG. 2, the apparatus 100 of the invention may be provided in a configuration that allows for continuous treatment of liquid medium. In this aspect, the apparatus 10 includes at least one set of electrodes 20 that includes one silver anode 30 and a cathode of noncorrosive metal 40. The electrodes 20 extend into a cylindrical container 65. The cylindrical container 65 attaches to a continuous flow manifold 70. The continuous flow manifold 70 includes a liquid medium inlet 80 and a liquid medium output 90. Each set of electrodes 20 is electrically connected to a power transformer 60. As shown in FIG. 3, the apparatus 200 of the invention may be configured with solar panels 110.

In this aspect of the invention where continuous flow is desired, a liquid medium may be flowed into the apparatus 200. Voltage settings will depend upon desired flow rates and desired silver ion concentration and may be calculated using Faraday's Law.

Aqueous Medium

The method and apparatus of the present invention are effective for use in an aqueous medium. As used herein, “aqueous medium” may include water, such as well water and irrigation water, chlorinated water, such as water that might be in a swimming pool, and drinking water such as water used for watering of livestock. Aqueous medium may also include tissues culture medium and other bacterial culture mediums. In one aspect, liquid medium may also include agricultural formulations that contain fertilizers, pesticides, herbicides and mixtures thereof. In this aspect of the invention, silver ion may be electrically generated in a solution that is already being applied to plants.

Application of Liquid Medium Containing Silver Ions

One general approach for applying the electrically generated silver ion liquid medium of the present invention is illustrated in FIG. 3. In one aspect of the invention, at least about 15 ppm silver ion is electrically generated in a liquid medium such as water. The liquid medium containing the silver ions may then be applied to plants in accordance with normal watering schedules. For example, a give type of plant type in a given geographic location may have a specific watering schedule. The method of the present invention can be used in accordance with an existing watering schedule.

In another aspect of the invention, silver ions may be generated continuously, such as is shown by the apparatus in FIG. 2. The amount of voltage applied will depend upon the desired flow rate for the particular use. The voltage settings may be determined from Faraday's Law.

All forms of plants and vegetation may benefit from application of an aqueous medium containing electrically generated silver ion. Some example of plants benefitting from application of electrically generated silver ions include grapes, corn, soybeans, tobacco, vegetables, melons, trees and cactus.

Reduction of Microbial Numbers in Water Holding Containers

In another important aspect, the present invention provides a method for reducing microbial growth in a water holding container. The water holding container may be for example water tanks used for the watering of livestock and poultry or a swimming pool. Microbial growth refers to growth by any of the microbes as described herein and also refers to algae species which are known to grow in water tanks.

In accordance with the method of the present invention, silver ions are electrically generated in the water container using the apparatus of the invention. Silver ions are generated in the water contained in the water holding container in an amount effective for reducing microbial growth in the water. In this aspect of the invention, the concentration of silver ions will be at least about 15 ppm silver ion.

The method of the invention is effective for reducing microbial numbers in the water by at least about one log, and in an important aspect, by at least about three logs or more. Silver ions generated in the water are not toxic to animals drinking the water and reduce or eliminate tank fouling, thereby reducing the need to drain and clean tanks. The use of the method in connection with swimming pools reduces or eliminates the need to use chlorine or other chemicals.

Regeneration of Plants in Tissue Culture

In another aspect, the present invention provides a method for regeneration of plants in tissue culture. In accordance with the method, plant cells are introduced into a tissue culture medium. The tissue culture medium contains at least about 15 ppm electrically generated silver ion. The silver ion level is maintained at a concentration of at least about 15 ppm, by periodically electrically generating silver ion. The method is effective for providing regenerated plants that are free of plant pathogens.

The following examples illustrate methods for carrying out the invention and should be understood to be illustrative of, but not limiting upon, the scope of the invention which is defined in the appended claims.

### EXAMPLES

### Example 1

### Antimicrobial Activity of Silver Ions

Antimicrobial activity of electrically generated silver ions was evaluated with Staphylococcus aureus and Pseudomonas aeruginosa. Microbes were inoculated into water an microbial counts were made at zero time. Silver ions were electrically generated in the water containing the microbes. Counts were performed at one hour. Results were as follows.

| | 0 Time | 1 Hour | |
|---|---|---|---|
| TEST ORGANISM | (CFU/ml) | (CFU/ml) | % Reduction |
| Staphylococcus aureus | 9,300 | <10 | 99.89 |
| Pseudomonas aeruginosa | 27,000 | <10 | 99.96 |
| Staphylococcus aureus | 10,000 | <10 | 99.90 |
| Pseudomonas aeruginosa | 27,000 | <10 | 99.96 |

### Example 2

### Treatment of Fungal and Bacterial Infections in Cactus

San Pedro cactus (Trichocereus pachanoi) and Blue Agave succulent (Blue Agave Tequilian) that were infected with fungus (Usarium oxisporum) and bacteria (Ervinia caratavora). Water containing 300 ppm electrically generated silver ion was applied to the plants three times a week. Within about 30 days from the start of treatment, plants displayed signs of new growth of about 1 inch. No new growth occurred in infected plants and healthy plants not treated with electrically generated silver ions.

### Example 3

### Treatment of Fungal Infections in Soybeans

Soybeans having infections of smut fungus were topically treated with water containing electrically generated silver ions twice a week and one watering. All visible signs of fungus infection were eliminated after 17 days. Microscopic analysis showed no fungal infection and plants treated with water containing electrically generated silver ion had noticeably more growth than healthy plants that had not been treated.

### Example 4

### Treatement of Fungal Infections in Squash

Squash infected with fungus were treated with water containing electrically generated silver ions twice a week and one watering. All visible signs of fungus infection were eliminated after 19 days. Microscopic analysis showed no fungal infection and plants treated with water containing electrically generated silver ion had noticeably more growth than healthy plants that had not been treated.

### Example 5

### Treatement of Bacterial Infections in Grapes

Chardonnay grapvines infected with E. coli and Xylella fastidosa were continuously treated with water containing electrically generated silver ions at a rate of three gallons per day every other day for 33 days. All visible signs of bacterial infection were eliminated after 33 days. Microscopic analysis showed no bacterial infection and plants treated with water containing electrically generated silver ion had noticeably more growth than healthy plants that had not been treated.

### Example 6

### Regeneration of Plants in Tissue Culture Medium

Three identical arrangements of apparatus were set-up to the foregoing description. The holes or apertures in the Pyrex test tubes were made by piercing the tubes with a butane torch. Silver wires were inserted through the holes in each of the tubes, and the protective tubes or sleeves were of silicone rubber. The protective tubes were sealed to the glass test tubes with Dow Corning type “A” medical adhesive. The silver wires defining the cathodes in each arrangement had a diameter of 0.020 inch an were formed into the bottom of each test tube in a single loop. The silver wires defining the anodes in each arrangement had a diameter of 0.010 inch and were brought to the center of each test tube and terminated in a small loop. The battery voltage was 6.0 volts and the resistor magnitude 2.7 megohms.

Each apparatus arrangement was sterilized in a steam autoclave, and then a nutrient medium was introduced into each test tube in an amount sufficient to cover the anode loop. The nutrient medium was Murashiga shoot multiplication medium, available from Grand Island Biological Co. under the number 500-119. A microscopic sample of Ficus elastica (rubber plant) was infected with a gram negative bacteria and placed on the anode loop. The electrical current into the anode wire was approximately 2 microamperes d.c.

Previous preparations without the foregoing electronic excitation had shown overnight growth of bacterial colonies about the clone. The three examples with electronic excitation showed no bacterial clouds. Their growth continued on to produce disease free plants. The foregoing evidences laboratory production of disease free offspring from infected plant stock by the method and apparatus of the present invention.

### Example 7

### Regeneration of Contaminated Plants in Tissue Culture Medium

The procedure of Example 6 was repeated, using one millimeter of leaf tissue taken from a Ficus elastica (rubber plant) known to be infected by an unknown bacteria. All previous attempts to get an uninfected clone from this parent had failed. The clone was placed in the nutrient solution resting on the anode loop of the pure silver wire. About one microampere of positive current was delivered to the anode for 24 hours. On repeated tests, currents ranging from 0.1 to 10 microamperes were used. In ten trials, the uninfected daughters were cloned from the infected parent. No appreciable difference was seen at the different current levels.

### Example 8

### Comparison of Tissue Culture Regeneration Methods

Seven samples of Ficus elastica (rubber plant) from a parent known to be infected were set up in nutrient agar solution at 37° C. In particular, group A consisted of three plant clones in test tubes with silver wires, electrically stimulated as described in Example 6. Group B consisted of two plant clones in test tubes set up as in Example 6, but with old wires, i.e. not stimulated at this time but previously stimulated at some earlier time. Group C consisted of two plant clones in test tubes with no electrodes and therefore served as a control group. Electrical current was supplied Group A according to the procedure of Example 6 for 92.5 hours. When the electrical current was turned off, the control group showed contamination and both groups A and B were clear. Seven days after the electrical current was turned off, all three groups showed contamination. Thus, the presence of stimulated or formerly stimulated electrodes delayed the appearance of bacteria well beyond its appearance in the control group.

### Example 9

### Inhibition of Gram Negative Bacterial Growth

Bacteria from Ficus elastica in the form of a gram negative rod of unknown type were cultered into agar onto a Petri dish provided with five silver wire anodes and a common silver wire cathode. The results are summarized in Table I as follows:

TABLE I

| Electrode | Measured Anode | | Anode |
|---|---|---|---|
| Number | Current @ 25.degree. C. | Results | Color |
| 1 | 2.479 uA | cleared area | black |
| | | 22 mm. times. 10 mm | |
| 2 | 0.734 uA | cleared area | black |
| | | 15 mm. times. 10 mm | |
| 3 | 0.732 uA | cleared area | black |
| | | 17 mm. times. 10 mm | |
| 4 | 0 | no cleared area | bright |
| 5 | 0 | no cleared area | bright |

As indicated the currents are in microamperes. Cleared area indicated killing of bacteria, and lack of clearing indicated no killing of bacteria. Thus the germicidal effect on these unknown plant bacteria is confirmed.

### Example 10

### Treatment of Bacterial Infections in Raspberry

The procedure of Example 9 was repeated using bacteria from infected raspberry plants. This was a gram negative diplococci of unknown type. The results were the same as for Example 9. All three stimulated electrodes killed the bacteria and the residual effects from unstimulated, used electrodes also killed the bacteria. The unstimulated, new electrodes, i.e. electrodes nos. 4, and 5, showed no cleared area indicating no killing.

### Example 11

### Treatment of Virus Infections in Chrysanthemum

Using an apparatus arrangement similar to the other examples above, a clone from a chrysanthemum known to be infected with a viroid (chrysanthemum stunt) was introduced into a modified Murashiga shoot media (GIBCO#500-1124) previously introduced to the container 10, with the clone resting on the pure silver anode wire loop 20. The cathode was a large-area pure silver helix rather than a single loop as shown in the drawing. About one microampere was passed through the silver anode all during the two-week growth period of the clone. The plant grew well and developed leaves and roots. At the conclusion of this time, the clone was pulverized introduced into a gel and subjected to gel electrophoresis. No viroid band was seen, suggesting that the silver ion environment had killed the viroid pathogen. This is a preliminary result, subject to confirmation by repetition.

### Example 12

### Killing of Bacteria

Glass petri dishes were prepared by drilling six to eight holes through the sides with CO₂ laser or with a butane micro-torch. A pure silver anode wire, insulated by a silicone sheath, was inserted through each hole and sealed in place with silicone cement such as Dow Corning medical adhesive “A”. The wires were 0.010 inch in diameter. Two cm of the length of each wire extended beyond the silicone sheath. A large-area central helix of pure silver having a diameter of 0.020 inch and a length of about 10 cm served as a common cathode. Each anode wire was connected through a current-limiting resistor to the positive terminal of six volt battery. This provided a different level of current to each anode. One or two anodes were always left unconnected, i.e. zero current, as controls.

The dishes were sterilized by autoclave and then filled about 5 mm deep with a sterile agar preparation. An animal bacteria culture was then introduced and allowed to grow for 24 hours at 37° C., producing a semi opaque cloud of bacterial colonies. With some trials new clean wires were used and the battery was connected after bacterial growth was complete. With others, the battery was connected immediately upon inoculation of the media. With still others, used dishes were cleared of media, washed, autoclaved and refilled with new media. The current was measured with a digital microammeter in some cases and calculated in others from voltage and resistor data, making suitable allowance for some voltage polarization loss at the metal/media interface.

The results were as follows: With new wires, cleared areas (killed bacteria) developed within 24 hours out to 5 mm from each stimulated anode. No clearing developed about the cathode, no clearing developed about new unstimulated anodes. Residual clearing developed about previously stimulated anodes which were rerun a second time in new media. Some clearing was observed as low as 25 nanoamperes. More clearing was developed by higher currents. Above 100 nanoamperes only modestly larger areas were cleared. At 1000 nanoamperes only about 10% more area was cleared as compared to 100 nanoamperes. When stimulation was applied immediately upon innoculation, area within 5 mm of stimulated anodes remained clear.

Based upon these results, it is concluded that bactericidal action seemed due to Ag+ ion, was confined to stimulated anodes. Bactericidal action was more or less linear up to 100 nanoamperes (with the 0.01″D×2 cm long electrodes) but nearly independent of current above that. Some bactericidal action was seen at a current level as low as 25 nanoamperes. Thus, the method and apparatus of the present test is useful in the electrical killing of animal bacteria by anodically generated silver ions using current levels as low as 25 nanoamperes.

I claim:

1. A method for treating and preventing infections in plants caused by plant pathogens, the method comprising:
electrically generating silver ions in an aqueous medium; and
applying aqueous medium containing silver ions to a plant in an amount effective for reducing infections in the plant caused by plant pathogens and for preventing infections in the plant caused by plant pathogens.

2. A method for treating and preventing infections in plants according to claim 1 wherein the plant pathogen is a bacteria, fungus, virus or mixtures thereof.

3. A method for treating and preventing infections in plants according to claim 1 wherein the silver ions are generated by electrical anodic corrosion of a silver wire in the aqueous medium.

4. A method for treating and preventing infections in plants according to claim 1 wherein the aqueous medium containing silver ion contains at least about 15 ppm silver ion.

5. A method for treating and preventing infections in plants according to claims 2 wherein the plant pathogen is a bacteria selected from the group consisting of Pseudomonas syringae, Pseudomonas viridiflava, Xanthomonas campestris pv. asclepiadas, Xyella fastidiosa, Acidovorax albilineans, and Acidovorax avenae sspl citrulli, E. coli, Erwinia amylovora and Ralstonia solanacearum.

6. A method for treating and preventing infections in plants according to claim 2 wherein the plant pathogen is Xylella fastidiosa.

7. A method for treating and preventing infections in plants according to claim 2 wherein the plant pathogen is leaf roll virus.

8. A method for treating and preventing infections in plants according to claim 2 wherein the plant pathogen is a fungus selected from the group consisting of Alternaria, Ascochyta, Botrytis, Cercospora, Colletotrichum, Diplodia, Erysiphe, Fusarium, Gaeumanomyces, Helminthosporium, Macrophomina, Nectria, Peronospora, Phoma, Phymatotrichum, Phytophthora, Plasmopara, Podosphaera, Puccinia, Pythium, Pyrenophora, Pyricularia, Rhizoctonia, Scerotium, Sclerotinia, Septoria, Thielaviopsis, Uncinula, Venturia, and Verticillium.

9. A method for treating and preventing infections in plants according to claim 2 wherein the plant pathogen is a virus selected from the group consisting of tobacco mosaic virus (TMV), cucumber mosaic virus (CMV), cucumber green mottle mosaic virus (as CGMMV), potato virus X (PVX), lettuce mosaic virus (LMV), and melon necrotic spot virus (MNSV).

10. A method for treating and preventing infections in plants according to claim 1 wherein the silver ions are generated by electrical anodic corrosion of a silver wire and the aqueous medium containing silver ions is applied to plants through an irrigation system.

11. A method for treating and preventing infections in plants according to claim 1 wherein the liquid medium is water.

12. A method for treating and preventing infections in plants according to claim 1 wherein the liquid medium is tissue culture medium.

13. A method for treating and preventing Pierce's Disease in plants, the method comprising:
electrically generating silver ions in an aqueous medium; and
applying aqueous medium containing silver ions to a plant in an amount effective for reducing Pierce's Disease in the plant and for preventing Pierce's Disease in the plant.

14. A method for treating and preventing Pierce's Disease in plants according to claim 13 wherein the aqueous medium containing silver ion contains at least about 15 ppm silver ion.

15. A method for treating and preventing Pierce's Disease in plants according to claim 13 wherein the silver ions are generated by electrical anodic corrosion of a silver wire and the aqueous medium containing silver ions is applied to plants through an irrigation system.

16. A method for treating and preventing Pierce's Disease in plants according to claim 13 wherein the liquid medium is water.

17. A method for treating and preventing Pierce's Disease in plants according to claim 13 wherein the liquid medium is tissue culture medium.

18. A method for treating and preventing Pierce's Disease in plant according to claim 13 wherein the plant is grape.

19. A method for reducing microbial growth in a water holding container, the method comprising electrically generating silver ions in water contained in the water holding container in an amount effective for reducing microbial growth in the water.

20. A method for reducing microbial growth in a water holding container according to claim 19 wherein the microbial growth is from bacteria, fungus, virus, algae or mixtures thereof.

21. A method for reducing microbial growth in a water holding container according to claim 19 wherein the silver ions are generated by electrical anodic corrosion of a silver wire in the aqueous medium.

22. A method for reducing microbial growth in a water holding container according to claim 19 wherein the electrical generation of silver ion is effective for providing water containing at least about 15 ppm silver ion.

23. A method for reducing microbial growth in a water holding container according to claim 19 wherein microbial numbers in the water are reduced by at least about one log.

24. A method for reducing microbial growth in a water holding container according to claim 19 wherein microbial numbers in the water are reduced by at least about three logs.

25. A method for regeneration of plants in tissue culture, the method comprising:
growing plant cells in a liquid medium, the liquid medium containing at least about 15 ppm electrically generated silver, and
maintaining a level of at least about 15 ppm electrically generated silver,
wherein regenerated plants are free of plant pathogens.

26. A method for regeneration of plants according to claim 25 wherein the plant pathogen is a bacteria, fungus, virus or mixtures thereof.

27. A method for regeneration of plants according to claim 25 wherein the silver ions are generated by electrical anodic corrosion of a silver wire in the aqueous medium.

28. A method for regeneration of plants according to claims 26 wherein the plant pathogen is a bacteria selected from the group consisting of Pseudomonas syringae, Pseudomonas viridiflava, Xanthomonas campestris pv. asclepiadas, Xyella fastidiosa, Acidovorax albilineans, and Acidovorax avenae sspl citrulli, E. coli, Erwinia amylovora and Ralstonia solanacearum.

29. A method for regeneration of plants according to claim 26 wherein the plant pathogen is a fungus selected from the group consisting of Alternaria, Ascochyta, Botrytis, Cercospora, Colletotrichum, Diplodia, Erysiphe, Fusarium, Gaeumanomyces, Helminthosporium, Macrophomina, Nectria, Peronospora, Phoma, Phymatotrichum, Phytophthora, Plasmopara, Podosphaera, Puccinia, Pythium, Pyrenophora, Pyricularia, Rhizoctonia, Scerotium, Sclerotinia, Septoria, Thielaviopsis, Uncinula, Venturia, and Verticillium.

30. A method for regeneration of plants according to claim 26 wherein the plant pathogen is a virus selected from the group consisting of tobacco mosaic virus (TMV), cucumber mosaic virus (CMV), cucumber green mottle mosaic virus (as CGMMV), potato virus X (PVX), lettuce mosaic virus (LMV), and melon necrotic spot virus (MNSV).

31. A method for treating and preventing leaf roll virus in plants, the method comprising:
electrically generating silver ions in an aqueous medium; and
applying aqueous medium containing silver ions to a plant in an amount effective for reducing leaf roll virus in the plant and for preventing leaf roll virus in the plant.

32. A method for treating and preventing leaf roll virus in plants according to claim 31 wherein the aqueous medium containing silver ion contains at least about 15 ppm silver ion.

33. A method for treating and preventing leaf roll virus in plants according to claim 31 wherein the silver ions are generated by electrical anodic corrosion of a silver wire and the aqueous medium containing silver ions is applied to plants through an irrigation system.

34. A method for treating and preventing leaf roll virus in plants according to claim 31 wherein the liquid medium is water.

35. A method for treating and preventing leaf roll virus in plants according to claim 31 wherein the liquid medium is tissue culture medium.

36. A method for treating and preventing leaf roll virus in plant according to claim 31 wherein the plant is grape.
